(19) United States
(12) Patent Application Publication
(10) Pub. No.: US 2026/0114952 A1
(43) Pub. Date: Apr. 30, 2026

(54) METHOD AND APPARATUS FOR DETECTING SURGICAL INFORMATION

(71) Applicant: METAL INDUSTRIES RESEARCH AND DEVELOPMENT CENTRE, Kaohsiung (TW)

(72) Inventors: Hsien-Ju WU, Kaohsiung (TW); Yun-Chen ZHANG, Kaohsiung (TW); Wen-Hui HUANG, Kaohsiung (TW); Bing-Feng HUANG, Kaohsiung (TW)

(21) Appl. No.: 18/965,922

(22) Filed: Dec. 2, 2024

(30) Foreign Application Priority Data
Oct. 28, 2024 (TW) 113141059

(51) Int. Cl. A61B 90/00; A61B 17/04; G01L 5/04

(52) U.S. Cl. CPC A61B 90/06; A61B 17/0401; G01L 5/042; A61B 2017/0404; A61B 2090/064

(57) ABSTRACT

A method for detecting surgical information is to be implemented by a wearable detection device. The wearable detection device is configured to detect a sensor chip that is disposed in a suture button for surgery. The method includes: detecting an ambient noise so as to generate an ambient sensing signal; detecting the sensor chip so as to generate a tension sensing signal; subtracting the ambient sensing signal from the tension sensing signal so as to generate a plurality of tension differences corresponding respectively to a plurality of scanning frequencies; and selecting one of the plurality of scanning frequencies that corresponds to a largest one of the plurality of tension differences as a resonance frequency, and obtaining a suture tension value based on the resonance frequency.

### CROSS-REFERENCE TO RELATED APPLICATION

This application claims priority to Taiwanese Invention Patent Application No. 113141059, filed on Oct. 28, 2024, the entire disclosure of which is incorporated by reference herein.

### FIELD

The disclosure relates to a method and an apparatus for detecting surgical information, and more particularly to a method and an apparatus for detecting surgical information to assist in medical monitoring.

### BACKGROUND

As medical technology advances, the general public increasingly pursues a healthier lifestyle, making the tracking of personal health information an important topic in modern times. For example, rotator cuff tendon inflammation can progress to a rotator cuff tear, and is more likely to occur in the elderly, athletes, labor workers and workers who spend a long time in specific postures (such as operating computers). A large population may be at risk for this health issue, and the number of patients seeking treatment for this health issue could gradually increase in the foreseeable future. Since surgical treatment is required for severe cases of this health issue, implanting a sensor chip in a patient for monitoring the tension of a suture in real time to track healing progress has become a valuable clinical technology.

However, several technical bottlenecks still exist in a conventional sensor chip. For example, a detection device that reads the conventional sensor chip is highly sensitive to ambient noise, which can lead to inaccurate measurements. Moreover, since the conventional sensor chip is required to be implanted close to the affected area, if the conventional sensor chip is placed too deep within the body, the detection range of the detection device may be insufficient, thereby further affecting the accuracy of the measurements.

### SUMMARY

Therefore, an object of the disclosure is to provide a method and an apparatus for detecting surgical information that can alleviate at least one of the drawbacks of the prior art.

According to an aspect of the disclosure, a method for detecting surgical information is to be implemented by a wearable detection device. The wearable detection device is configured to detect a sensor chip that is disposed in a suture button for surgery. The method includes: detecting an ambient noise so as to generate an ambient sensing signal; detecting the sensor chip so as to generate a tension sensing signal; subtracting the ambient sensing signal from the tension sensing signal so as to generate a plurality of tension differences corresponding respectively to a plurality of scanning frequencies; and selecting one of the plurality of scanning frequencies that corresponds to a largest one of the plurality of tension differences as a resonance frequency, and obtaining a suture tension value based on the resonance frequency.

According to another aspect of the disclosure, an apparatus for detecting surgical information includes a sensor chip that is disposed in a suture button for surgery, and a wearable detection device. The wearable detection device is configured to detect an ambient noise so as to generate an ambient sensing signal, and detect the sensor chip so as to generate a tension sensing signal. The wearable detection device is further configured to subtract the ambient sensing signal from the tension sensing signal so as to generate a plurality of tension differences corresponding respectively to a plurality of scanning frequencies, select one of the plurality of scanning frequencies that corresponds to a largest one of the plurality of tension differences as a resonance frequency, and obtain a suture tension value based on the resonance frequency.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating an apparatus for detecting surgical information according to an embodiment of the disclosure.

FIG. 2 is a circuit diagram illustrating some components of a signal calibration unit of a control module of the apparatus according to an embodiment of the disclosure.

FIG. 3 is a block diagram illustrating a detection unit of a detection module of the apparatus according to an embodiment of the disclosure.

FIG. 4 is a flow chart illustrating a noise detection procedure of a method for detecting surgical information according to an embodiment of the disclosure.

FIG. 5 is a flow chart illustrating a sensor chip detection procedure of the method for detecting surgical information according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

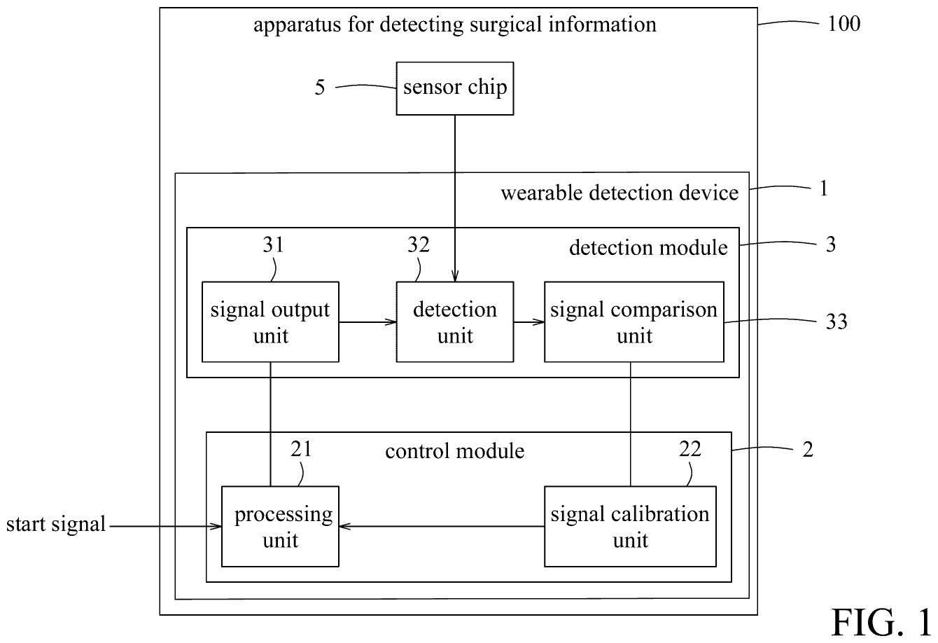

Referring to FIG. 1, an apparatus 100 according to an embodiment of the disclosure is configured to implement a method for detecting surgical information. The method includes a noise detection procedure and a sensor chip detection procedure. The apparatus 100 includes a sensor chip 5 and a wearable detection device 1.

The sensor chip 5 is disposed in a suture button in the body of a patient for surgery. In this embodiment, the sensor chip 5 may be implemented by an LC resonant circuit, but the disclosure is not limited to such. The sensor chip 5 includes a parallel plate capacitor, and when the suture button is subjected to an external force (e.g., is pulled by a suture during surgery), a distance between two ends of the parallel plate capacitor changes accordingly. The change in distance alters a capacitance of the parallel plate capacitor, thereby affecting a frequency at which the sensor chip 5 resonates.

The wearable detection device 1 is configured to detect an ambient noise and the sensor chip 5, and includes a control module 2 and a detection module 3 that are electrically connected to each other. The control module 2 includes a processing unit 21, and a signal calibration unit 22 that has a device ambient signal, an ambient threshold and a tension threshold pre-stored therein.

In this embodiment, the processing unit 21 may be implemented by a microprocessor unit (MPU) or a microcontroller unit (MCU). The signal calibration unit 22 may use algorithms (coded in a programming or scripting language such as C, C++, Java, assembly languages, and Python) implemented through a combination of data structures, programs, routines, or other programming configurations, or may be realized through various numbers of hardware configurations that perform specific functions, but the disclosure is not limited to such.

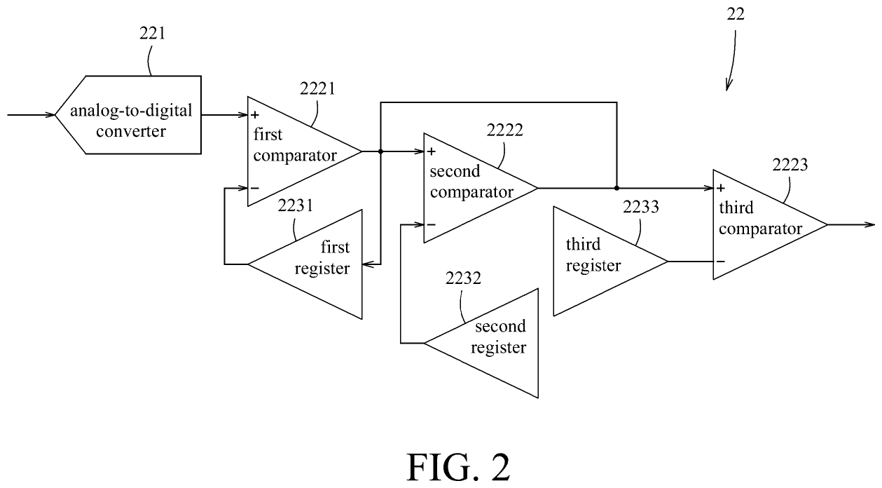

Referring further to FIG. 2, in an embodiment where the signal calibration unit 22 is realized through hardware configurations, the signal calibration unit 22 is electrically connected to the processing unit 21, and includes an analog-to-digital converter (ADC) 221, a first comparator 2221, a second comparator 2222, a third comparator 2223, a first register 2231, a second register 2232 and a third register 2233. The first register 2231 is configured to store a signal outputted by the first comparator 2221, the second register 2232 is configured to store the device ambient signal, and the third register 2233 is configured to store the ambient threshold and the tension threshold.

A positive input terminal of the first comparator 2221 and a negative input terminal of the first comparator 2221 are electrically connected to an output terminal of the ADC 221 and an output terminal of the first register 2231, respectively. A positive input terminal of the second comparator 2222 and a negative input terminal of the second comparator 2222 are electrically connected to an output terminal of the first comparator 2221 and an output terminal of the second register 2232, respectively. The output terminal of the first comparator 2221 is electrically connected to an input terminal of the first register 2231. A positive input terminal of the third comparator 2223 and a negative input terminal of the third comparator 2223 are electrically connected to an output terminal of the second comparator 2222 and an output terminal of the third register 2233, respectively. The positive input terminal of the third comparator 2223 is further electrically connected to the output terminal of the first comparator 2221. In one embodiment, signal transmission between the abovementioned components is controlled by the processing unit 21 in conjunction with other electronic components, such as a multiplexer and/or a demultiplexer.

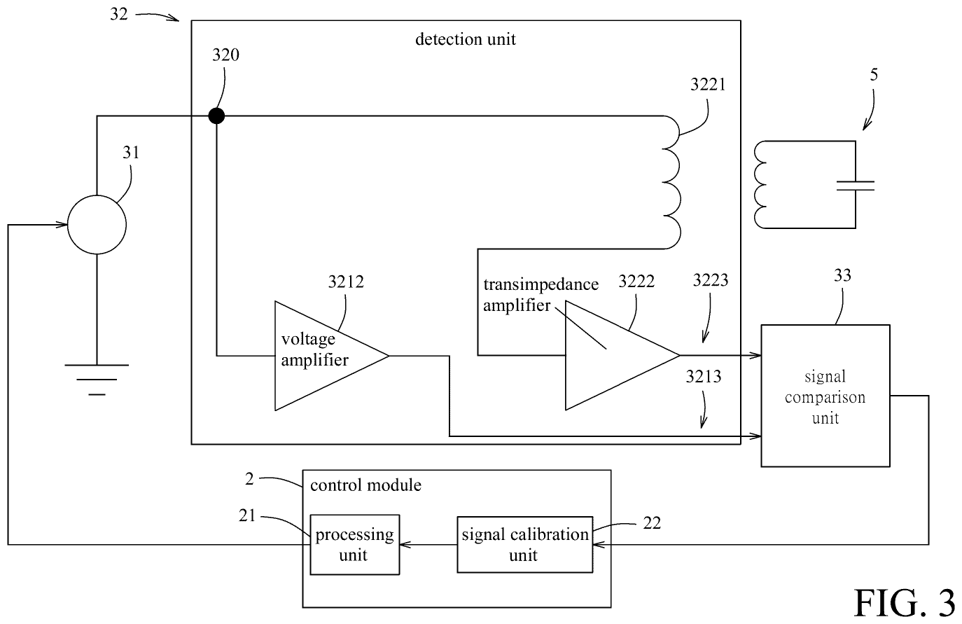

Referring further to FIG. 3, the detection module 3 includes a signal output unit 31 that is electrically connected to the processing unit 21, a detection unit 32 that is electrically connected to the signal output unit 31, and a signal comparison unit 33 that is electrically connected to the signal calibration unit 22 and the detection unit 32. In this embodiment, the signal output unit 31 is a voltage-controlled oscillator (VCO), the detection unit 32 is realized through various numbers of hardware configurations that perform specific functions, and the signal comparison unit 33 is a phase detector, but the disclosure is not limited to such.

In this embodiment, the detection unit 32 includes a detection input terminal 320 that is electrically connected to the signal output unit 31, a first output terminal 3213 and a second output terminal 3223 that are electrically connected to the signal comparison unit 33, a voltage amplifier 3212, and a detection circuit 3221 and a transimpedance amplifier 3222 that are connected in series. The detection input terminal 320, the voltage amplifier 3212 and the first output terminal 3213 collectively form a first transmission path. The detection input terminal 320, the detection circuit 3221, the transimpedance amplifier 3222 and the second output terminal 3223 collectively form a second transmission path. The detection circuit 3221 is configured to detect the ambient noise and the sensor chip 5. The voltage amplifier 3212 and the transimpedance amplifier 3222 each are configured to amplify a signal received thereby.

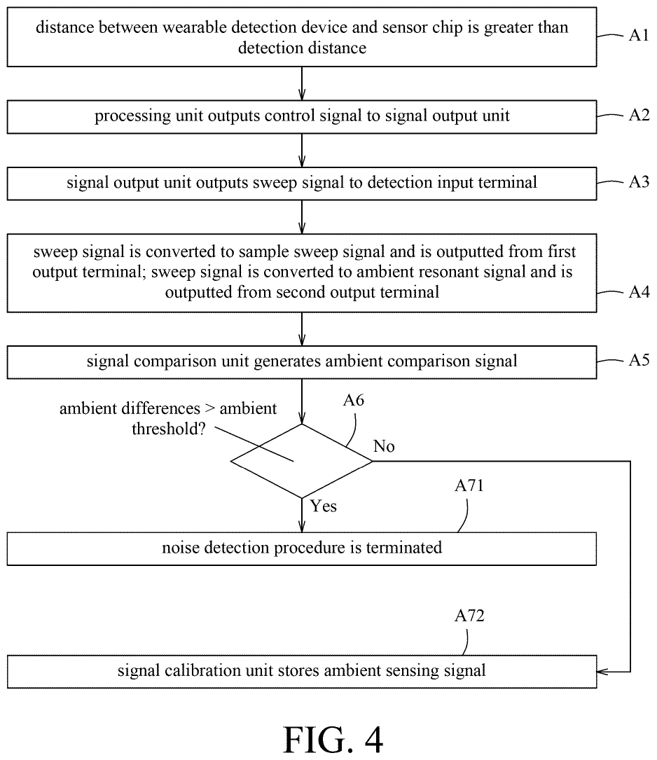

Referring further to FIG. 4, the method for detecting surgical information includes the noise detection procedure, which includes steps A1 to A72.

In step A1, the wearable detection device 1 is disposed such that a distance between the wearable detection device 1 and the sensor chip 5 is greater than a detection distance of the wearable detection device 1. It should be noted that the detection distance depends on sensitivity of the wearable detection device 1.

In step A2, in response to receiving a start signal, the processing unit 21 outputs a control signal to the signal output unit 31 based on the start signal, where a signal type of the control signal is a voltage signal. The start signal may be generated by a user operating an input device (e.g., a key or a button on the wearable detection device 1) that is electrically connected to the processing unit 21.

In step A3, the signal output unit 31 outputs a sweep signal to the detection input terminal 320 of the detection unit 32 based on the control signal, where the sweep signal covers a plurality of scanning frequencies, and a signal type of the sweep signal is an analog signal.

In step A4, the sweep signal enters the first transmission path and the second transmission path through the detection input terminal 320 of the detection unit 32. In the first transmission path, the voltage amplifier 3212 amplifies an amplitude of the sweep signal and converts the sweep signal thus amplified to a sample sweep signal, and the sample sweep signal is outputted from the first output terminal 3213. In the second transmission path, when the sweep signal passes through the detection circuit 3221, the detection circuit 3221 detects the ambient noise based on the sweep signal so as to generate an ambient sweep signal that covers the scanning frequencies, where a signal type of the ambient sweep signal is a current signal. To describe in further detail, when the sweep signal passes through the detection circuit 3221, any interference source in the environment that produces LC resonance may cause phase changes in the sweep signal at certain scanning frequencies, which is recorded in the ambient sweep signal. In the second transmission path, the transimpedance amplifier 3222 amplifies an amplitude of the ambient sweep signal and converts the ambient sweep signal thus amplified to an ambient resonant signal, where a signal type of the ambient resonant signal is a voltage signal, and the ambient resonant signal is outputted from the second output terminal 3223.

In step A5, the signal comparison unit 33 receives the sample sweep signal and the ambient resonant signal from the first output terminal 3213 and the second output terminal 3223, respectively, and compares the sample sweep signal and the ambient resonant signal so as to generate an ambient comparison signal. Specifically, the signal comparison unit 33 detects a phase difference and an amplitude difference between the sample sweep signal and the ambient resonant signal, so as to generate the ambient comparison signal, and sends the ambient comparison signal to the signal calibration unit 22, where a signal type of the ambient comparison signal is an analog signal.

In step A6, the signal calibration unit 22 converts the ambient comparison signal to an ambient sensing signal that covers the scanning frequencies, and obtains a plurality of ambient differences corresponding respectively to the scanning frequencies, where the ambient differences are between the ambient sensing signal and the device ambient signal that is stored in the control module 2. The signal calibration unit 22 further determines whether any of the ambient differences is greater than the ambient threshold. When the determination in step A6 is affirmative, a flow of the method proceeds to step A71; otherwise, the flow of the method proceeds to step A72.

In the embodiment shown in FIG. 2 where the signal calibration unit 22 is realized through hardware configurations, the ADC 221 of the signal calibration unit 22 receives the ambient comparison signal, converts the ambient comparison signal into the ambient sensing signal, which has a signal type of a digital signal, and sends the ambient sensing signal to the first comparator 2221. At this time, since the first register 2231 is empty, the first comparator 2221 directly sends the ambient sensing signal to the second comparator 2222. The second comparator 2222 subtracts the device ambient signal that is stored in the second register 2232 from the ambient sensing signal, so as to generate the ambient differences corresponding respectively to the scanning frequencies. The third comparator 2223 compares each of the ambient differences to the ambient threshold that is stored in the third register 2233 so as to determine whether any of the ambient differences is greater than the ambient threshold. When any of the ambient differences is greater than the ambient threshold, the flow of the method proceeds to step A71; otherwise, when none of the ambient differences is greater than the ambient threshold, the flow of the method proceeds to step A72.

In a case where at least one of the ambient differences is greater than the ambient threshold, it means that there is an interference source that exists in the environment and that may affect accuracy of the detection module 3 in detecting signals. Therefore, in step A71, the noise detection procedure is terminated, and the user should remove the interference source from the environment for more accurate measurements taken by the detection module 3.

In another case where none of the ambient differences is greater than the ambient threshold, it means that no interference source, which may affect accuracy of the detection module 3 in detecting signals, exists in the environment. Therefore, in step A72, in the embodiment shown in FIG. 2, the first comparator 2221 sends the ambient sensing signal to the first register 2231, and the first register 2231 stores the ambient sensing signal.

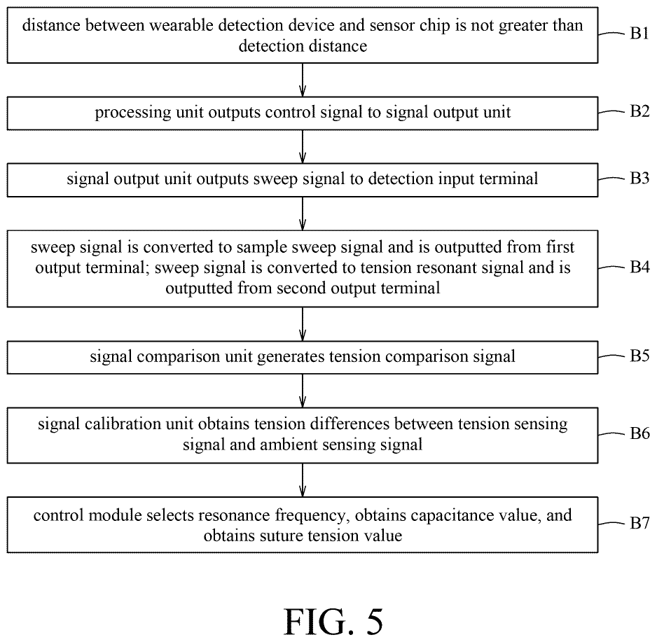

Referring further to FIG. 5, the method for detecting surgical information includes the sensor chip detection procedure, which includes steps B1 to B7.

In step B1, the wearable detection device 1 is disposed such that the distance between the wearable detection device 1 and the sensor chip 5 is not greater than the detection distance of the wearable detection device 1.

In step B2, in response to receiving the start signal, the processing unit 21 outputs the control signal to the signal output unit 31 based on the start signal.

In step B3, the signal output unit 31 outputs the sweep signal to the detection input terminal 320 of the detection unit 32 based on the control signal.

In step B4, the sweep signal enters the first transmission path and the second transmission path through the detection input terminal 320 of the detection unit 32. In the first transmission path, the voltage amplifier 3212 amplifies the amplitude of the sweep signal and converts the sweep signal thus amplified to the sample sweep signal, and the sample sweep signal is outputted from the first output terminal 3213. In the second transmission path, when the sweep signal passes through the detection circuit 3221, the detection circuit 3221 detects the sensor chip 5 based on the sweep signal so as to generate a tension sweep signal that covers the scanning frequencies, where a signal type of the tension sweep signal is a current signal. To describe in further detail, when the sweep signal passes through the detection circuit 3221, the LC resonance produced by the sensor chip 5 may cause phase changes in the sweep signal at certain scanning frequencies, which is recorded in the tension sweep signal. In the second transmission path, the transimpedance amplifier 3222 amplifies an amplitude of the tension sweep signal and converts the tension sweep signal thus amplified to a tension resonant signal, where a signal type of the tension resonant signal is a voltage signal, and the tension resonant signal is outputted from the second output terminal 3223.

In step B5, the signal comparison unit 33 receives the sample sweep signal and the tension resonant signal from the first output terminal 3213 and the second output terminal 3223, respectively, and compares the sample sweep signal and the tension resonant signal so as to generate a tension comparison signal. Specifically, the signal comparison unit 33 detects a phase difference and an amplitude difference between the sample sweep signal and the tension resonant signal, so as to generate the tension comparison signal, and sends the tension comparison signal to the signal calibration unit 22, where a signal type of the tension comparison signal is an analog signal.

In step B6, the signal calibration unit 22 converts the tension comparison signal to a tension sensing signal that covers the scanning frequencies, and obtains a plurality of tension differences corresponding respectively to the scanning frequencies, where the tension differences are between the tension sensing signal and the ambient sensing signal. In the embodiment shown in FIG. 2, the ADC 221 of the signal calibration unit 22 receives the tension comparison signal, converts the tension comparison signal into the tension sensing signal, which has a signal type of a digital signal, and sends the tension sensing signal to the first comparator 2221. The first comparator 2221 subtracts the ambient sensing signal that is stored in the first register 2231 from the tension sensing signal so as to generate the tension differences corresponding respectively to the scanning frequencies.

In step B7, the control module 2 selects one of the scanning frequencies that corresponds to a largest one of the tension differences as a resonance frequency, obtains a capacitance value of the sensor chip 5 based on the resonance frequency, and obtains a suture tension value based on the capacitance value. It is noted that a resonance frequency of an LC resonant circuit may be represented as a function of an inductance value and a capacitance value of the LC resonant circuit. Moreover, a capacitance value of a capacitor is related to a distance between two terminals of the capacitor, which may be changed when a force (e.g., the suture tension value) is applied to the two terminals. Since calculations for obtaining the capacitance value and the suture tension value are well known to one having ordinary skill in the art, they will not be described in further detail for the sake of brevity.

In the embodiment shown in FIG. 2, the first comparator 2221 sends the tension differences to the third comparator 2223. The third comparator 2223 compares the tension differences to the tension threshold that is stored in the third register 2233 so as to determine whether any of the tension differences is greater than the tension threshold. In a case where none of the tension differences is greater than the tension threshold, it means that the detection circuit 3221 fails to successfully detect the sensor chip 5, and another attempt for the wearable detection device 1 to detect the sensor chip 5 should be made. In another case, upon the third comparator 2223 determining that one of the tension differences is greater than the tension threshold, the processing unit 21 selects the one of the tension differences as the largest one of the tension differences. Then, the processing unit 21 selects one of the scanning frequencies that corresponds to the one of the tension differences as the resonance frequency, obtains the capacitance value of the sensor chip 5 based on the resonance frequency, and obtains the suture tension value based on the capacitance value. As such, during a surgery, the suture tension value may provide information for a doctor to adjust tension of the suture, and after the surgery, the suture tension value may be used for tracking healing progress of the patient.

In summary, according to the disclosure, the user may operate the apparatus 100 for detecting surgical information. The detection module 3 of the wearable detection device 1 detects the ambient noise and the sensor chip 5 so as to generate the ambient comparison signal and the tension comparison signal, respectively. The control module 2 of the wearable detection device 1 converts the ambient comparison signal and the tension comparison signal to the ambient sensing signal and the tension sensing signal, respectively, and subtracts the ambient sensing signal from the tension sensing signal so as to obtain the tension differences. The control module 2 then selects one of the scanning frequencies that corresponds to the largest one of the tension differences as the resonance frequency, and obtains the capacitance value and the suture tension value based on the resonance frequency. As such, the disclosure addresses the problem of the wearable detection device 1 being highly sensitive to the ambient noise, and increases the reliability of the measurements taken by the wearable detection device 1.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for detecting surgical information to be implemented by a wearable detection device, the wearable detection device being configured to detect a sensor chip that is disposed in a suture button for surgery, the method comprising:
detecting an ambient noise so as to generate an ambient sensing signal;
detecting the sensor chip so as to generate a tension sensing signal;
subtracting the ambient sensing signal from the tension sensing signal so as to generate a plurality of tension differences corresponding respectively to a plurality of scanning frequencies; and
selecting one of the plurality of scanning frequencies that corresponds to a largest one of the plurality of tension differences as a resonance frequency, and obtaining a suture tension value based on the resonance frequency.

2. The method as claimed in claim 1, the wearable detection device storing a device ambient signal and an ambient threshold, wherein the detecting an ambient noise includes:
in response to receiving a sweep signal that covers the plurality of scanning frequencies, detecting the ambient noise based on the sweep signal so as to generate the ambient sensing signal that covers the plurality of scanning frequencies;
obtaining a plurality of ambient differences corresponding respectively to the plurality of scanning frequencies and between the ambient sensing signal and the device ambient signal; and
in a case where none of the plurality of ambient differences is greater than the ambient threshold, storing the ambient sensing signal.

3. The method as claimed in claim 1, wherein when detecting the ambient noise, the wearable detection device is disposed such that a distance between the wearable detection device and the sensor chip is greater than a detection distance.

4. The method as claimed in claim 1, wherein the detecting the sensor chip includes, in response to receiving a sweep signal that covers the plurality of scanning frequencies, detecting the sensor chip based on the sweep signal so as to generate the tension sensing signal that covers the plurality of scanning frequencies, and
wherein the obtaining a suture tension value includes obtaining a capacitance value of the sensor chip based on the resonance frequency, and obtaining the suture tension value based on the capacitance value.

5. The method as claimed in claim 1, wherein when detecting the sensor chip, the wearable detection device is disposed such that a distance between the wearable detection device and the sensor chip is not greater than a detection distance.

6. An apparatus for detecting surgical information, comprising:
a sensor chip that is disposed in a suture button for surgery; and
a wearable detection device that is configured to
detect an ambient noise so as to generate an ambient sensing signal,
detect said sensor chip so as to generate a tension sensing signal,
subtract the ambient sensing signal from the tension sensing signal so as to generate a plurality of tension differences corresponding respectively to a plurality of scanning frequencies, and
select one of the plurality of scanning frequencies that corresponds to a largest one of the plurality of tension differences as a resonance frequency, and obtain a suture tension value based on the resonance frequency.

7. The apparatus as claimed in claim 6, wherein said wearable detection device includes a control module and a detection module, and wherein:
said control module is configured to output a control signal to said detection module;
said detection module is configured to generate a sweep signal that covers the plurality of scanning frequencies in response to receiving the control signal, detect the ambient noise based on the sweep signal so as to generate an ambient comparison signal, and send the ambient comparison signal to said control module;
said control module is further configured to convert the ambient comparison signal to the ambient sensing signal that covers the plurality of scanning frequencies, and to obtain a plurality of ambient differences corresponding respectively to the plurality of scanning frequencies and between the ambient sensing signal and a device ambient signal that is stored in said control module; and
said control module is further configured to, in a case where none of the plurality of ambient differences is greater than an ambient threshold, store the ambient sensing signal.

8. The apparatus as claimed in claim 7, wherein said detection module includes a signal output unit, a detection unit and a signal comparison unit, and wherein:
said signal output unit is configured to receive the control signal, and to output the sweep signal based on the control signal;
said detection unit is configured to detect the ambient noise so as to generate an ambient resonant signal, and to send the sweep signal and the ambient resonant signal to said signal comparison unit; and
said signal comparison unit is configured to compare the sweep signal to the ambient resonant signal so as to generate the ambient comparison signal.

9. The apparatus as claimed in claim 7, wherein said control module includes a processing unit, and a signal calibration unit that is electrically connected to said processing unit and that includes an analog-to-digital converter (ADC), a first comparator, a second comparator, a third comparator, a first register, a second register and a third register, and wherein:
said first register is configured to store a signal outputted by said first comparator, said second register is configured to store the device ambient signal, and said third register is configured to store the ambient threshold;
a positive input terminal of said first comparator and a negative input terminal of said first comparator are electrically connected to an output terminal of said ADC and an output terminal of said first register, respectively;
an output terminal of said first comparator is electrically connected to an input terminal of said first register;
a positive input terminal of said second comparator and a negative input terminal of said second comparator are electrically connected to the output terminal of said first comparator and an output terminal of said second register, respectively;
a positive input terminal of said third comparator and a negative input terminal of said third comparator are electrically connected to an output terminal of said second comparator and an output terminal of said third register, respectively; and
the positive input terminal of said third comparator is electrically connected to the output terminal of said first comparator.

10. The apparatus as claimed in claim 6, wherein said wearable detection device includes a control module and a detection module, and wherein:
said control module is configured to output a control signal to said detection module;
said detection module is configured to generate, in response to receiving the control signal, a sweep signal that covers the plurality of scanning frequencies, to detect said sensor chip based on the sweep signal so as to generate a tension comparison signal, and to send the tension comparison signal to said control module; and
said control module is further configured to convert the tension comparison signal to the tension sensing signal that covers the plurality of scanning frequencies, obtain a capacitance value of said sensor chip based on the resonance frequency, and obtain the suture tension value based on the capacitance value.
